(19) United States
(12) Patent Application Publication
(10) Pub. No.: US 2007/0167808 A1
(43) Pub. Date: Jul. 19, 2007

(54) ULTRASOUND PROBE FOR PARACENTESIS AND ULTRASOUND DIAGNOSTIC APPARATUS

(76) Inventor: Mitsuhiro Nozaki, Tokyo (JP)

Correspondence Address: Patrick W. Rasche;Suite 2600, Armstrong Teasdale LLP, One Metropolitan Square, St. Louis, MO, 63102, US

(21) Appl. No.: 11/564,920

(22) Filed: Nov. 30, 2006

(30) Foreign Application Priority Data
Dec. 9, 2005 (JP) 2005-356755

(51) Int. Cl. A61B 8/14

(52) U.S. Cl. 600/459

(57) ABSTRACT

An ultrasound probe for paracentesis with a biopsy needle attached to a probe body, which permits an operator to insert the biopsy needle into an exact position of a patient, is to be provided. An ultrasound probe for paracentesis comprising a probe body and a biopsy needle attached to the probe body, wherein the center of an acoustic elements array provided in the probe body is offset toward the biopsy needle attached to the probe body with respect to a central vertical line of the probe body.

### BACKGROUND OF THE INVENTION

The present invention relates to an ultrasound probe for paracentesis and an ultrasound diagnostic apparatus having the ultrasound probe for paracentesis.

Paracentesis has been conducted wherein a biopsy needle such as an injection needle is inserted into a living body to sample a tumor tissue for example or for local administration of a drug. For avoiding damage of a blood vessel which would cause a large quantity of bleeding or for positive paracentesis of a tumor tissue for example, the paracentesis is usually performed while making reference to a sectional image formed by an ultrasound diagnostic apparatus.

As methods for performing the paracentesis while being guided by the ultrasound diagnostic apparatus there are known a method wherein an ultrasound probe for paracentesis is used and a biopsy needle is inserted into a living body through a guide hole formed centrally of a probe surface (serving not only as an ultrasound irradiation surface but also as an ultrasound detecting surface) which is a surface for contact with the living body and a method wherein an adapter for biopsy needle is attached to an ultrasound probe for diagnosis and a biopsy needle supported by the adapter for biopsy needle is inserted into a living body from near an end portion of a probe surface as a surface for contact with the living body.

As to the ultrasound probe for paracentesis used in the former method wherein a biopsy needle is inserted into a living body through a guide hole formed centrally of the probe surface, a sectional image can be photographed from a central part of the probe, i.e., from above a central vertical line of the probe body. Consequently, there accrues an advantage such that a clear image is obtained, the biopsy needle can be inserted into a living body from above a central line of a grip part of the probe body while seeing a clear sectional image, and the biopsy needle can be inserted safely into the living body. However, it is impossible to collect image data of the portion positioned just under the biopsy needle guide hole formed centrally of the probe surface, so there occurs a dropout portion (dead angle) of image near the central part of the upper side of the sectional image and thus the method in question is not suitable for other ultrasound inspections than paracentesis. Therefore, for performing the conventional ultrasound inspection, it is necessary to separately provide an ultrasound probe for diagnosis, which is uneconomical.

For such a reason, in many cases there actually is adopted the latter method wherein an adapter for biopsy needle is attached to an ultrasound probe for diagnosis and paracentesis is performed using a biopsy needle supported by the adapter (see, for example, Patent Literature 1).

This mode of the adapter for biopsy needle (simply as “adapter” hereinafter as the case may be) being attached to the ultrasound probe for diagnosis (simply as “ultrasound probe” hereinafter) is illustrated in FIG. 1 which is a citation of a drawing of Patent Literature 1. FIG. 1 is a perspective view showing an assembled state of both probe 101 and adapter 2. As ultrasound probes for paracentesis there are a convex type using a curved probe surface 13 and a linear type using a planar probe surface. The illustrated probe is the convex type.

As shown in FIG. 1, the adapter 2 is fixed detachably to a body 111 of the ultrasound probe 101, the position of a biopsy needle 3 is prescribed onto a scanning axis of the ultrasound probe 101, and the biopsy needle 3 is displayed at all times on a sectional image formed in an ultrasound diagnostic apparatus.

In such a construction, the biopsy needle 3 is guided by a hole 21 formed in the adapter 2 for biopsy needle and is inserted into a living body from near an end portion of the probe surface 13 of the ultrasound probe 101.

Japanese Patent Laid-Open Publication No. 2004-147984.

The ultrasound probe 101 is connected to an ultrasound diagnostic apparatus (not shown) through a probe cable 14, and on a display prepared in the ultrasound diagnostic apparatus there are displayed a tomographic image 5 and a paracentetic marker 51, as shown in FIG. 2 for example. Since an expected insertion route of the biopsy needle 3 is displayed by the paracentetic marker 51, an operator can insert the biopsy needle 3 accurately into, for example, a desired tumor tissue while avoiding a blood vessel which is likely to cause a large quantity of bleeding. The angle of the biopsy needle 3 relative to the surface of a living body is determined by a sensor (not shown) and an expected insertion route of the biopsy needle 3 is determined, then the paracentetic marker 51 is displayed on the surface of the image 5 automatically.

FIG. 2 is an explanatory diagram of a section of the conventional ultrasound probe 101 and a picture plane photographed by the probe 101. The biopsy needle 3 is attached to an adapter (not shown), which in turn is attached to the ultrasound probe body 111. An acoustic elements array 12 is provided at a tip of the ultrasound probe body 111. The acoustic elements array 12 comprises plural acoustic elements arranged in a convex shape. A central axis 15 of the acoustic elements array 12 passing through a central point P of the same array is disposed symmetrically right and left of the drawing with respect to a central vertical line 16 of the probe body (grip part) 111. The central axis 15 of the acoustic elements array serves as the center of a predetermined field angle at which the acoustic elements array radiates an ultrasound wave.

In performing paracentesis with use of such an apparatus, scanning is conducted while changing the angle of the ultrasound probe 101 relative to a living body as necessary. However, when the adapter 2 is attached to the probe body 111, the angle of inclination of the probe body (grip part) 111 toward the adapter 2 is restricted by the adapter 2, with a consequent likelihood that there occurs a partial disturbance in the tomographic image 5 positioned on the adapter 2 side, as shown in FIG. 2, leading to formation of a dead angle 52 as an indistinct portion of the image 5. Thus, there has been the problem that the biopsy needle cannot be positioned accurately while looking at the image.

### SUMMARY OF THE INVENTION

Therefore, it is an object of the present invention to provide an ultrasound probe for paracentesis which permits an operator to insert a biopsy needle into an exact position of a patient, as well as an ultrasound diagnostic apparatus using such an ultrasound probe for paracentesis.

For solving the above-mentioned problem, the ultrasound probe for paracentesis according to the present invention comprises a probe body and a biopsy needle attached to the probe body, wherein the center of an acoustic elements array provided in the probe body is offset to the biopsy needle side attached to the probe body with respect to a central vertical line of the probe body.

The ultrasound diagnostic apparatus according to the present invention with an ultrasound probe for paracentesis comprises a probe body and a biopsy needle attached to the probe body, wherein the center of an acoustic elements array provided in the probe body of the ultrasound probe for paracentesis is offset to the biopsy needle side attached to the probe body with respect to a central vertical line of the probe body.

In the ultrasound probe for paracentesis according to the present invention, since the center of the acoustic elements array is positioned on the biopsy needle side attached to the probe, the biopsy needle can be inserted into an exact position while looking at a clear image. In the ultrasound diagnostic apparatus according to the present invention, the biopsy needle of the ultrasound probe for paracentesis attached to the apparatus can be inserted into an exact position while looking at a clear image.

Further objects and advantages of the present invention will be apparent from the following description of the preferred embodiments of the invention as illustrated in the accompanying drawings.

### BRIEF DESCRIPTION OF THE DRAWINGS

FIG. 1 is a perspective view showing conventional ultrasound probe and adapter for biopsy needle;

FIG. 2 is an explanatory diagram showing an ultrasound wave emitting direction from an acoustic elements array in the conventional ultrasound probe;

FIG. 3 is a sectional view showing an ultrasound probe and an adapter for biopsy needle according to the present invention;

FIG. 4 is an explanatory diagram showing an ultrasound wave emitting direction from an acoustic elements array in the ultrasound probe of the present invention; and

FIG. 5 is a block diagram of an ultrasound diagnostic apparatus using the ultrasound probe of the present invention.

### DETAILED DESCRIPTION OF THE INVENTION

An ultrasound probe for paracentesis according to an embodiment of the present invention will be described hereinunder concretely with reference to the drawings.

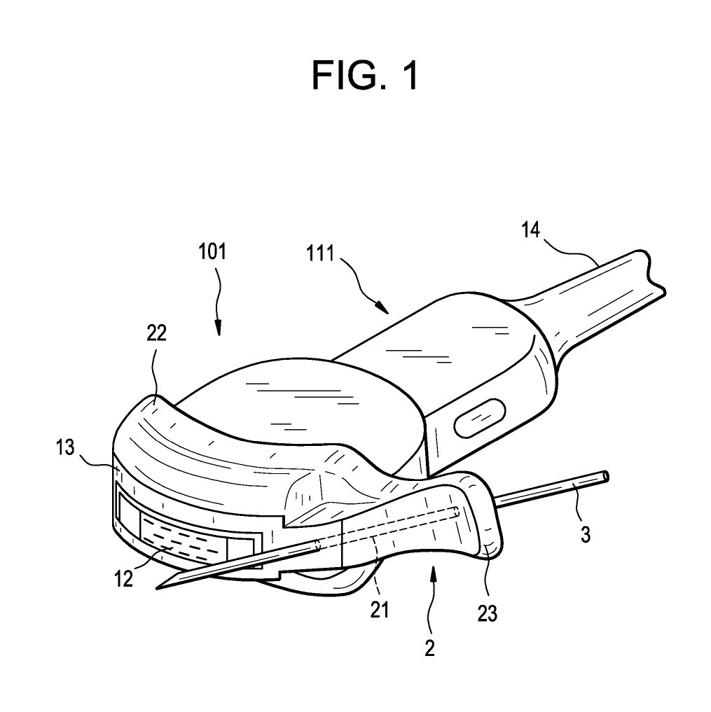

FIG. 3 is a sectional view of an ultrasound wave-generating tip portion of the ultrasound probe 1 for paracentesis, showing an assembled state of a probe body 11 and an adapter 2. FIG. 3 is a sectional view of the tip portion of the ultrasound probe 1 for paracentesis, but an entire perspective view of the probe is omitted because it is common to the conventional ultrasound probe shown in FIG. 1.

As shown in FIG. 3, the adapter 2 for biopsy needle for the ultrasound probe is attached to the ultrasound probe 1 detachably. The adapter 2 is made up of an attaching/detaching portion 22 capable of being attached to and detached from the probe body 11 and a prescribing portion 23 for prescribing a biopsy needle 3 onto an ultrasound scanning line by the ultrasound probe 1. The attaching/detaching portion 22 of the adapter 2 is inserted and fixed into the probe body 11 of the ultrasound probe 1, a hole 21 which permits insertion therein of the biopsy needle 3 is formed in the prescribing portion 23 and the biopsy needle 3 is inserted through the hole 21, then the adapter 2 is attached to the ultrasound probe 1, whereby positioning of the biopsy needle 3 is performed.

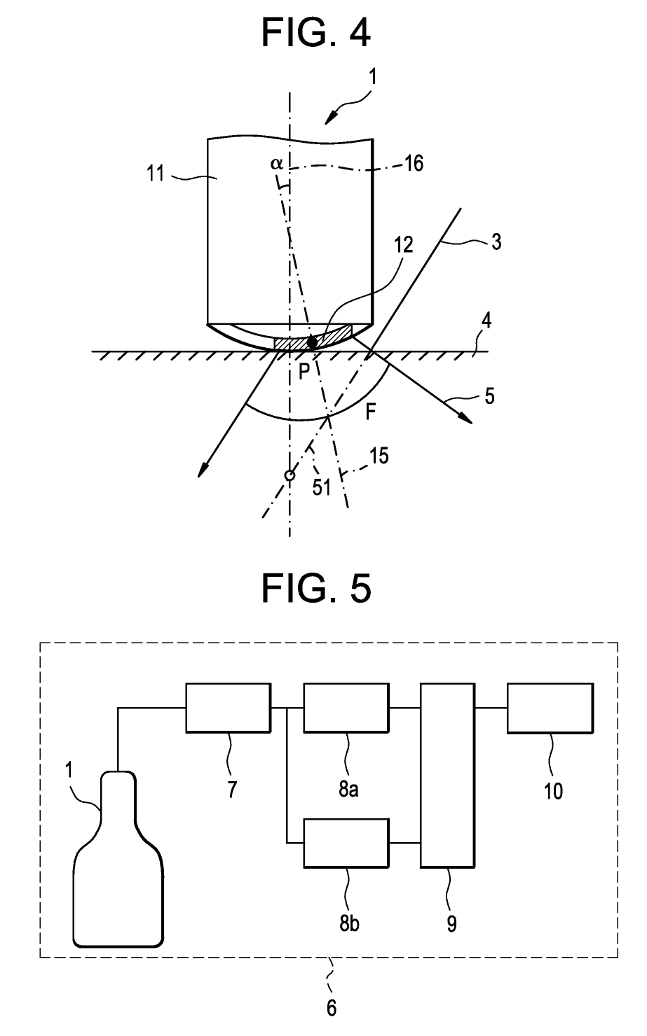

In the case of thus attaching the adapter 2 for biopsy needle to the ultrasound probe body 11 and performing paracentesis, when the ultrasound probe 1 is brought into contact with the surface of a living body 4 and an image obtained is observed, a tomographic image 5 and a paracentetic marker 51 are displayed as shown in FIG. 4, thus indicating an insertion path of the biopsy needle 3, so that the operator can insert the biopsy needle 3 in accordance with the paracentetic marker 51.

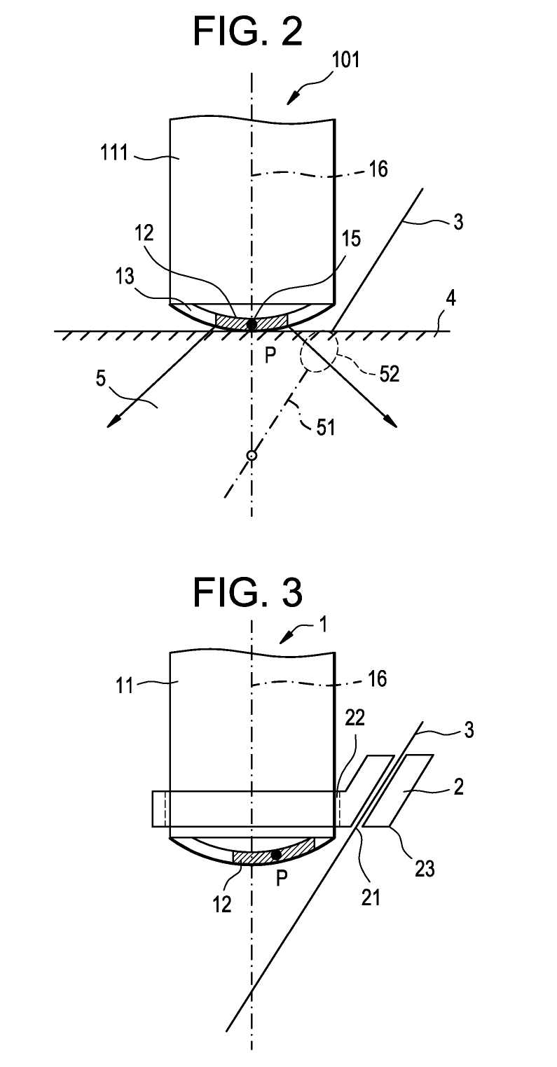

For searching for an affected part of a patient, the operator causes the body of the patient to move while grasping the probe body 11 of the probe 1 or causes the axis of the probe body 11 to be tilted. When the probe body 11 is tilted to search for the affected part, the angle of inclination of the probe body is restricted by the adapter 2 attached to the probe body 11 and as the case may be there is formed a dead angle 52 (see FIG. 2) in searching for the affected part, thus obstructing the insertion of the biopsy needle 3.

The present invention eliminates the aforesaid dead angle. In the ultrasound probe 1 of this embodiment, as shown in FIG. 3 and FIG. 4 which shows the arrangement of an acoustic elements array 12 at the probe tip and an emitting direction of an ultrasound wave emitted from the acoustic elements array 12, a central point P of the acoustic elements array 12 is offset toward the biopsy needle 3 attached to the probe with respect to a central vertical line 16 of the probe body 11. More specifically, as shown in FIG. 4, a central axis 15 of the acoustic elements array 12 as an ultrasound wave emitting array passing through the central point P of the same array which point P serves as a scanning center of the array is inclined toward the adapter 2 (toward the biopsy needle 3) by a certain angle α from the vertical line 16 of the probe body (grip part) 11. In FIG. 4, the angle of the central axis 15 of the acoustic elements array from the central vertical line 16 of the probe body 11 is set at about 15°. The scanning center generally corresponds to a bisector of the angle at which the acoustic elements array 12 emits an ultrasound wave with a predetermined field angle.

By thus tilting the central axis 15 of the acoustic elements array at an angle of α toward the biopsy needle 3 from the vertical line 16 of the probe body 11, even if the angle of inclination of the probe body (grip part) 11 is restricted by the adapter 2, it becomes possible to photograph the image 5 so as to eliminate a dead angle between the position of the biopsy needle 3 attached to the adapter 2 and the image 5 obtained in the acoustic elements array 12. That is, the dead angle 52 shown in FIG. 2 is eliminated and the image obtained becomes very clear. Consequently, paracentesis can be performed accurately and safely without missing the affected part and without imposing any burden on the affected part.

A method of using a very large number of acoustic elements and increasing the number of channels may be effective in eliminating the dead angle of image and obtaining a clear image. However, increasing the number of channels results in the structure of the diagnostic apparatus becoming complicated and hence increase of expenses, leading to an increase of device cost, which is not advisable also in point of maintenance.

In the ultrasound probe according to the present invention, by using a minimum number of acoustic elements required, exact data (image) can be offered to the operator, a dead angle in the inserting direction of the biopsy needle 3 can be eliminated, and a clear image can be obtained in the inserting direction of the biopsy needle 3. That is, in the present invention, the central position of the acoustic elements array 12 is inclined toward the biopsy needle attached to the probe without changing the number of channels in the array 12 from that in the existing probe. Thus, in the ultrasound probe 1 according to the present invention, while keeping the number of channels in the acoustic elements array 12 equal to that in the existing ultrasound probe, the center of the array 12 is shifted toward the biopsy needle attached to the probe, whereby the clearness of image in the inserting direction of biopsy needle is improved and hence it is possible to grasp an affected part clearly and insert the biopsy needle safely.

The angle α of inclination of the direction of the scanning center of the acoustic elements array 12 (the direction of the central axis 15 of the same array) from the central vertical line 16 of the probe body 11 cannot be determined sweepingly, depending on for example the condition of the patient's body and the position of the living body 4 into which the biopsy needle 3 is inserted. However, as an example, given that the field angle at which the acoustic elements array 12 emits an ultrasound wave is F, it is desirable that the design is made so as to satisfy the following relationship:
0<90°−(F/2+α)<15°

This is because, by so doing, even when the number of channels in the acoustic elements array 12 is decreased resulting in the field angle F becoming smaller, the dead angle in the inserting direction of the biopsy needle 3 can be eliminated by setting large the angle α of inclination of the central direction of the acoustic elements array 12 from the central vertical line 16 of the probe body. That is, as the angle α becomes larger, an end of the field angle F comes to overhang the biopsy needle 3.

By thus changing the central axis 15 of the acoustic elements array 12 passing through the central point P of the same array, the dead angle is eliminated as in FIG. 4 and a needle inserting position is clearly displayed, thereby permitting an accurate and safe insertion of the needle. The paracentetic marker 51 shown in FIG. 4 determines an expected route of insertion of the biopsy needle 3 on the basis of an angle of the same needle relative to the surface of the living body which angle is detected by a sensor (not shown), and is displayed on the image 5 automatically.

An example of an ultrasound diagnostic apparatus having the ultrasound probe 1 described above will be described with reference to FIG. 5. An ultrasound diagnostic apparatus 6 shown in FIG. 5 includes the ultrasound probe 1, a transmitter/receiver 7, a B-mode signal generator 8a, a D-mode signal generator 8b, a DSC (Digital Scan Converter) 9, and a display unit 10.

The ultrasound diagnostic apparatus 6 illustrated in FIG. 5 diagnoses a subject by utilizing the Pulse Doppler method. In the ultrasound diagnostic apparatus 6, the transmission of an ultrasound wave to the subject from the ultrasound probe 1 and the reception of an echo signal after being transmitted to and reflected by the subject are performed through the transmitter/receiver 7 connected to the ultrasound probe 2. The B-mode signal generator 8a generates data for B mode on the basis of the echo signal received from the transmitter/receiver 7. The D-mode signal generator 8b determines a change in frequency of reflected ultrasound pulses on the basis of the echo signal received in the ultrasound probe 2 and a signal of ultrasound pulses transmitted toward the subject, and generates a Doppler signal.

The DSC 9 generates a B-mode image on the basis of the data for B mode generated by the B-mode signal generator 8a or generates a D-mode image on the basis of the Doppler signal generated by the D-mode signal generator 8b and causes the thus-generated images to be displayed on the display unit 10. In the ultrasound diagnostic apparatus 6 described above, the ultrasound probe for paracentesis usually employed in the ultrasound diagnostic apparatus is replaced by the ultrasound probe 1 for paracentesis according to the present invention.

In the ultrasound diagnostic apparatus shown in FIG. 5 there is used the ultrasound probe 1 for paracentesis wherein the center of the acoustic elements array 12 provided in the probe body 11 is offset toward the biopsy needle 3 attached to the probe from the central vertical line of the probe body 11.

Therefore, the operator of the ultrasound diagnostic apparatus 6 can insert the biopsy needle 3 of the ultrasound probe 1 attached to the apparatus into an exact position safely while looking at a clear image displayed on the display unit 10 of the apparatus.

Although reference has been made above to the convex-shaped elements array, the present invention is applicable also to a linear elements array. The profile shape of the ultrasound probe and that of the adapter related to the present invention are arbitrary and modifications may be made insofar as they do not depart from the scope of the present invention.

Many widely different embodiments of the invention may be configured without departing from the spirit and the scope of the present invention. It should be understood that the present invention is not limited to the specific embodiments described in the specification, except as defined in the appended claims.

1. An ultrasound probe for paracentesis, comprising:
a probe body; and
a biopsy needle attached to the probe body,
wherein the center of an acoustic elements array provided in the probe body is offset to the biopsy needle side attached to the probe body with respect to a central vertical line of the probe body.

2. An ultrasound probe for paracentesis according to claim 1, wherein the acoustic elements array is disposed in a convex shape.

3. An ultrasound probe for paracentesis according to claim 1, further comprising an attaching/detaching portion capable of being attached to and detached from the probe body, the attaching/detaching portion having a prescribing portion formed with a hole,
wherein the biopsy needle is inserted through the hole of the prescribing portion and is thereby attached to the probe body.

4. An ultrasound probe for paracentesis according to claim 1, wherein, given that a field angle at which the acoustic elements array emits an ultrasound wave is F and the angle between a scanning center direction of the acoustic elements array and the central vertical line of the probe body is α, the following relationship is satisfied:
0<90°−(F/2+α)<15°

5. An ultrasound diagnostic apparatus provided with an ultrasound probe for paracentesis comprising:
a probe body; and
a biopsy needle attached to the probe body,
wherein the center of an acoustic elements array provided in the probe body of the ultrasound probe for paracentesis is offset to the biopsy needle side attached to the probe body with respect to a central vertical line of the probe body.

6. An ultrasound diagnostic apparatus according to claim 5, wherein the acoustic elements array of the ultrasound probe is disposed in a convex shape.

7. An ultrasound diagnostic apparatus according to claim 5, wherein the ultrasound probe further comprises an attaching/detaching portion capable of being attached to and detached from the probe body, the attaching/detaching portion having a prescribing portion formed with a hole, and the biopsy needle is inserted through the hole of the prescribing portion and is thereby attached to the probe body.

8. An ultrasound diagnostic apparatus according to claim 5, wherein, given that a field angle at which the acoustic elements array of the ultrasound probe emits an ultrasound wave is F and the angle between a scanning center direction of the acoustic elements array and a central vertical line of the probe body is α, the following relationship is satisfied:
0<90°−(F/2+α)<15°
